(19) United States
(12) Patent Application Publication
(10) Pub. No.: US 2002/0172938 A1
(43) Pub. Date: Nov. 21, 2002

(54) Adsorbing and non-adsorbing surfaces for biological materials

(76) Inventors: Jerome J. Cuomo, Raleigh, NC (US); Vinay Sakrani, Raleigh, NC (US); Stefan Ufer, Carrboro, NC (US)

Correspondence Address: MYERS BIGEL SIBLEY & SAJOVEC, PO BOX 37428, RALEIGH, NC, 27627, US

(21) Appl. No.: 09/826,431

(22) Filed: Apr. 4, 2001

(51) Int. Cl. C12Q 1/70; C12Q 1/68; G01N 33/53; G01N 33/567; G01N 33/542; C12M 1/34

(52) U.S. Cl. 435/5; 435/6; 435/7.2; 435/7.9; 435/287.2

(57) ABSTRACT

A substrate having a chemically modified surface comprises a base layer having a coating positioned thereon. The coating includes a chemically crosslinked material comprising elements selected from the group consisting of (1) M, O, C, H, and N; wherein M is a metal selected from the group consisting of silicon, titanium, tantalum, germanium, boron, zirconium, aluminum, hafnium and yttrium, (2) M, O, H, and N wherein M is defined above, (3) C, (4) O, C, H, and N, and (5) M or C and one of O, H, or N, wherein the chemically crosslinked material is terminated with at least one electrophilic or nucleophilic functional group.

### FIELD OF THE INVENTION

The invention generally relates to a substrate having a chemically modified surface that may be used in the adsorption and non-adsorption of various materials, and methods of forming and using the same.

### BACKGROUND OF THE INVENTION

Electrochemical systems are widely known in the art for detecting and analyzing a number of materials. In one example, aqueous sample solutions are capable of being analyzed for the presence and concentration of certain biomolecules. More specifically, in electrochemical detection systems for these biomolecules, electrochemical reactions taking place on the surface of chemically modified substrates can be evaluated.

The signal containing the desired analytical information is typically present in the form of an electrical current measured at a given potential difference applied between two or three electrodes, e.g., working, counter, and reference electrodes. Of all materials that may be present in a sample solution, biomolecules are capable of giving rise to an electrochemical current due to their contact with the electrode surface. A conventional electrode surface is typically only a fraction of the total surface area of the entire electrochemical cell which comes in contact with the detection medium. If even a small molecule to be measured adsorbs on a surface other than the surface of the working electrode, this non-specifically adsorbed biomolecule is lost for measurement. As a result, an artificially lowered current signal results. Since biomolecules can be measured electrochemically down to the femtomole-range, virtually any non-specific adsorption typically has a tremendous impact on the performance of such electrochemical detection systems.

In view of the above, there is a need in the art for chemically modified surfaces and methods of using the same which allow for selective adsorption and non-adsorption of various materials such as, for example, biomolecules.

### SUMMARY OF THE INVENTION

In one aspect, the invention provides a substrate having a chemically modified surface. The substrate comprises a base layer having a coating positioned thereon. The coating comprises a chemically crosslinked material comprising elements selected from the group consisting of (1) M, O, C, H, and N; wherein M is a metal selected from the group consisting of silicon, titanium, tantalum, germanium, boron, zirconium, aluminum, hafnium and yttrium; (2) M, O, H, and N wherein M is defined above; (3) C; (4) O, C, H, and N; and (5) M or C, and one of O, H, or N. The chemically crosslinked material has a surface which is terminated with at least one electrophilic or nucleophilic functional group.

In another aspect, the invention provides a detection system comprising a substrate described herein.

In another aspect, the invention provides a microfluidic system comprising a substrate described herein.

In another aspect, the invention provides a method of chemically modifying a surface on a substrate. The method comprises exposing a composition comprising at least one precursor to an energy source to form a energized precursor; promoting the energized precursor into an excited state to produce ionic materials; depositing the ionic materials on a base layer such that the ionic materials form a coating thereon. The coating comprises a chemically crosslinked material comprising elements selected from the group consisting of (1) M, O, C, H, and N; wherein M is a metal selected from the group consisting of silicon, titanium, tantalum, germanium, boron, zirconium, aluminum, hafnium and yttrium; (2) M, O, H, and N wherein M is defined above, (3) C; (4) O, C, H, and N; and (5) M or C, and one of 0, H, or N; and treating the chemically crosslinked material such that the material is terminated with at least one electrophilic or nucleophilic functional group

These and other aspects are encompassed by the invention and are described herein.

### DETAILED DESCRIPTION OF THE PREFERRED EMBODIMENTS

The present invention now will be described more fully hereinafter with reference to the accompanying specification, drawings, and examples, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

In one aspect, the invention provides a substrate having a chemically modified surface. The substrate comprises a base layer having a coating positioned thereon. The coating comprises a chemically crosslinked material comprising elements selected from the group consisting of (1) M, O, C, H, and N; wherein M is a metal selected from the group consisting of silicon, titanium, tantalum, germanium, boron, zirconium, aluminum, hafnium and yttrium; (2) M, O, H, and N wherein M is defined above; (3) C; (4) O, C, H, and N; and (5) M or C, and one of O, H, or N. The coating has a surface which is terminated with at least one electrophilic or nucleophilic functional group.

A number of base layers may be used according to the invention including, without limitation, metals, organic polymers, inorganic polymers, ceramics, glass, plastics, elastomers, and the like. The base layer may be electrically conducting or non-conducting as desired by one skilled in the art. Exemplary embodiments include, but are not limited to, carbon (e.g., graphite, glassy carbon, pyrolytic graphite, carbon paste, carbon fiber), along with platinum, gold, and palladium, alloys of the above, and combinations of the above. Doped and undoped oxides may also be used such as, without limitation, indium-doped tin oxide (ITO), tin oxide, titanium oxide, manganese oxide, and lead oxide. Semiconductor materials may be used including, without limitation, Si, Ge, ZnO, CdS, TiO₂, and GaAs. Alloys and combinations of the above can also be used. As stated above, the chemically crosslinked material may comprise a number of combination of elements. In one embodiment, M is silicon which may be present in combination with any one or more of the three elements: O, H, and N. In one preferred embodiment, the chemically crosslinked material is applied by a plasma chemical vapor deposition and results in a crosslinked amorphous, matrix. In one embodiment, the material comprises from about 5 to about 30 percent by weight silicon, about 10 to about 60 percent carbon, about 10 to about 60 percent hydrogen and trace amounts of oxygen, i.e., preferably less than 3 percent. In one preferred embodiment, the chemically crosslinked material comprises from about 30 to about 60 percent carbon and from about 10 to about 40 percent silicon. The coating may have a number of various thicknesses. For example, in one embodiment, the coating may have a thickness which is at least a fraction of a monolayer thick. For the purposes of the invention, the term “fraction” of a monolayer refers to at least about 50 percent by area of the patterned surface regions where the coating is intended to be deposited. In a preferred embodiment, the coating has a thickness ranging from about 200 nanometers to about 400 nanometers .

Any number of electrophilic functional groups can be used in the invention. For the purposes of the invention, the term “electrophilic functional group” may be defined as a functional group which has a electropositive polarity. Preferably, the electrophilic functional group comprises one or more elements selected from the group consisting of carbon, hydrogen, nitrogen and silicon. Exemplary electrophilic functional groups include, without limitation, any group capable of imparting a positive polarity to the coating material including, for example, hydrogen-terminations, alkyl groups, and quarternary ammonium group molecules.

Advantageously, a number of materials may be adsorbed to the electrophilic functional group. For the purposes of the invention, the term “adsorbed” can be defined as increased adhesion of dissolved materials (e.g., biomolecules) to the surface of the chemically crosslinked material of this invention. The term “non-adsorbed” may be defined as decreased adhesion of dissolved materials (e.g.,biomolecules) to the surface of the chemically crosslinked material. Not intending to be bound by theory, it is believed that a positive polarity is present on the coating surface which electrostatically attracts and adsorbs negatively charged groups. Exemplary groups include, for example, electron-rich biomolecules or polyanions such as, without limitation, DNA, nucleic acids, proteins, enzymes, cells, viruses, and combinations thereof. Examples of these materials are set forth in U.S. Pat. No. 5,968,745 to Thorp et al., the disclosure of which is incorporated herein by reference in its entirety.

In another embodiment, the surface of the coating may be terminated with at least one nucleophilic functional group. For the purposes of the invention, the term “nucleophilic functional group” is defined as a group capable of donating a pair of electrons to an electrophile. In various embodiments, the nucleophilic functional group comprises one or more elements selected from the group consisting of carbon, silicon, halogens, oxygen, hydrogen, nitrogen, sulfur, and phosphorus. Examples of electronegative surface sites include, without limitation, oxygenated or fluorinated functional groups or any other groups displaying an electronegative polarity.

Typically, the surface of the coating material having nucleophilic functional groups attached thereto is advantageous in that a build up of a negative polarity on the surface results. Accordingly, materials having a negative charge (e.g., biomolecules) are repelled, and thus the surface is non-adsorbing.

In one embodiment, various portions of the surface of the coating may be terminated with at least one electrophilic functional group while other surface portions may be terminated with at least one nucleophlic functional group. In a preferred embodiment, the portion of the surface of the coating terminated with at least one electrophilic functional group and the portion of the surface of the coating terminated with at least one nucleophilic functional group may be adjacent to one another. This embodiment is particularly desirable in that it allows for the selective formation of adsorbing and non-adsorbing regions on the substrate. A method of forming such surfaces is described in detail hereinbelow.

The substrate of the invention can be employed in a number of devices, the selection of which can be made by one who is skilled in the art. For example, the substrate can be used in a detection system such as, without limitation, an electrochemical detection system, a chemical detection system, and an optical detection system. In one embodiment, the substrate can also be employed in a microfluidic system such as one described in Ser. No. 09/625,903 filed Jul. 26, 2000, the disclosure of which is incorporated herein by reference in its entirety. As an example, in one embodiment, the microfluidic system may include at least one biofouling surface.

In another aspect, the invention provides a method of chemically modifying a surface on a substrate. The method comprises exposing a composition comprising at least one precursor to an energy source to form a energized precursor; promoting the energized precursor into an excited state to produce ionic materials; depositing the ionic materials on a base layer such that the ionic materials form a coating thereon. The coating comprising a chemically crosslinked material comprising elements selected from the group consisting of (1) M, O, C, H, and N; wherein M is a metal selected from the group consisting of silicon, titanium, tantalum, germanium, boron, zirconium, aluminum, hafnium and yttrium; (2) M, O, H, and N wherein M is defined above, (3) C; (4) O, C, H, and N; and (5) M or C, and one of 0, H, or N; and treating the chemically crosslinked material such that the material is terminated with at least one electrophilic or nucleophilic functional group.

The coating used in accordance with the invention can be deposited on a substrate using a variety of techniques, the selection of which is known to one skilled in the art. Exemplary techniques include, without limitation, Plasma Enhanced Chemical Vapor Deposition (PECVD), Chemical Vapor Deposition (CVD), sputtering, evaporation, and other deposition techniques such as plating, dip-, flow-, spray-, or spin coating. In a preferred embodiment, the coating is formed out of a material using precursors from, for example, silanes, siloxanes (e.g., polysiloxanes), silazanes, hydrocarbons, various metalorganics, titanates, or metal alkoxides, as well as combinations of any of the above. Examples of precursors that can be used include, without limitation, tetramethylsilane, trimethylsilane, tetramethoxysilane, hexamethyl disilane, hexamethyl disiloxane, hexamethyl disilazane, methane, ethane, ethylene, tetraalkoxy titanates, etc, as well as combinations thereof.

In one embodiment, a PECVD process may be employed utilizing any of the above-mentioned precursors (typically in the form of feed gas) introduced with or without argon or other inert gas and energizing these materials into a plasma by direct current, radio frequency, microwave, enhanced plasmas or by hollow cathode magnetron energy sources. The energized precursor (with or without energized argon or other inert gas) is promoted into an excited state, producing at least one of ionic fragments, free radicals, atoms, molecules, and mixtures thereof, in an excited state which bombard and reconstruct on a base layer to produce a coating described herein. Among other factors, the precursor and the process conditions are believed to influence the specific compositions and properties of the coatings. The plasma enhanced chemical vapor deposition (PECVD) preferably is carried out at or slightly above room temperature. However, it will be appreciated that other temperatures can be employed. For example, in one embodiment, a higher substrate temperature in the range from about 300 to about 400° C. may be used for deposition to attain specific properties such as lower incorporation of hydrogen in the coatings for potentially improved mechanical and temperature stability. Various pressures may be employed. For example, in one embodiment, the pressure may range from about 10 mTorr to about 1 atm (760 Torr). In the preferred embodiment a pressure of 50 mTorr was used.

After forming the coating on the base layer, the surface of the coating can be modified using various techniques. In other words, the treating step of the above method can encompass various embodiments. For example, in one embodiment, an electronegative post-treatment of a surface of a coating can be a plasma treatment using various substances such as, without limitation, oxygen, ozone, water vapor, nitrogen oxide, fluorine, or fluorine gases/gas mixtures including fluorocarbons, fluorosilanes, or any other gas that would result in functionalities having a negative polarity associated with them. The electronegative post-treatment results in the surface being terminated with at least one nucleophilic functional group. Another embodiment encompasses a chemical post-treatment of the surface utilizing, as an example, perfluorinated polyethers, silanes terminated with electronegative functionalitites, or similar compounds such as those typically used for self assembled monolayers (SAMs).

Electropositive post treatment of the surface of the coating may be carried out using a number of techniques including, but not limited to, a plasma treatment employing hydrogen. Chemical post-treatments to obtain electropositive polarities may be used as well if desired. The electropositive post-treatment results in the surface of the coating being terminated with at least one electrophilic functional group.

The substrate (and hence the coating thereon) is highly advantageous in that it does not decay over time. As an example, the coating does not decay for preferably a period of at least 1 year.

In one preferred embodiment, an electrically conducting intermediate layer may be present between the coating and the base layer.

In various embodiments, the intermediate layer may comprise one or more electrically conducting metals or metal alloys such as, without limitation, platinum, gold, and/or palladium, as well as one or more electrically conducting doped and undoped oxides such as, without limitation, indium-doped tin oxide (ITO), tin oxide, titanium oxide, manganese oxide, zinc oxide, and lead oxide, as well as materials including, without limitation, gold, platinum, palladium, carbon, silicon, germanium, cadmium sulfide, titanium dioxide, and gallium arsenide. Combinations and alloys of the above may also be employed.

In one aspect, the intermediate layer is typically used to form an electrode whose active electrode opening is defined by an opening in the coating while the connection leads that provide electrical contacts to the working electrode are encapsulated by the coating. In another aspect, the coating is capable of providing a non-adsorbing surface for various materials (e.g., large biomolecules), which otherwise may possibly be lost for electrochemical detection due to adsorption to surfaces surrounding the electrode openings.

The patterning of the intermediate layer may be carried out by employing a number of techniques such as, for example, wet etching, lift off, shadow masking, screen printing, and the like. These procedures are illustrated in greater detail herein.

The intermediate layer may be employed in such a manner to form a number of various patterns in combination with the coating and the base layer. For example, in one embodiment, the coating may be selectively removed such that openings are formed therein to allow the intermediate layer to be exposed. In another embodiment, the intermediate layer and the coating present thereon are removed in a manner to expose the base layer underneath the intermediate layer and coating layer. Alternatively, in another embodiment, the intermediate layer and the coating may be present such that the base layer is completely encapsulated.

The invention will now be described in greater detail with respect to the examples. It should be understood that the examples are for the purposes of illustration, and does not serve to limit the invention as defined by the claims.

### EXAMPLE 1

### Deposition of Plasma Enhanced (PECVD) Coating

A coating is deposited using a PECVD process by employing the following parameters and precursor gas.

Step 1: Substrate Precleaning

An argon and oxygen plasma treatment is used to clean the substrate surface prior to the deposition of the passivation layer. This step is intended to remove adsorbed organic impurities from the surface to allow for good adhesion of the coating. The following conditions are employed:

| | |
|---|---|
| Gas mixture: | 50 percent argon and 50 percent oxygen |
| Processing pressure: | 50 mTorr |
| Bias Voltage: | −250 V |
| Processing time: | 10 min |

Step 2: Passivation Layer

A passivation layer is deposited on the substrate employing the following conditions:

| | |
|---|---|
| Active precursor: | tetra methyl silane Si(CH₃)₄, 3 percent in argon |
| Processing pressure: | 50 mTorr |
| Bias voltage: | −250 V |

A coating thickness of approximately 300 nm results.

Step 3: Plasma Surface Activation

In this embodiment, a plasma activation is performed as the terminating step. It should be appreciated that this step is optional, depending on the surface adsorption properties desired. Thus, this embodiment is non-limiting. In an attempt to minimize or avoid non-specific adsorption of nucleic acids and proteins, an oxygen plasma treatment is performed to terminate the coating surface with functional groups having negative polarities. Nucleic acids being polyanions are believed to not adsorb to these negative functionalities, which is highly advantageous.

The following processing conditions are employed:

| | |
|---|---|
| Processing gas: | 100 percent oxygen |
| Processing pressure: | 50 mTorr |
| Bias voltage: | −250 V |
| Processing time: | 1 min. |

### EXAMPLE 2

### Patterning of Intermediate layer

Various embodiments for intermediate layer patterning are set forth below:

1. Wet etching:

a. Deposit and pattern photoresist as etch mask on top of blanket coated intermediate layer

b. Selectively etch intermediate layer by bringing exposed areas of intermediate layer in contact with chemical etchant

c. Remove etch mask

2. Lift-off

a. Deposit and pattern photoresist on top of surface of base layer

b. Vacuum deposit material for intermediate layer on top of base layer

c. Remove photoresist and thereby lifting off portions of intermediate layer, which were deposited on top of photoresist leaving patterned intermediate layer behind

3. Shadow masking:

patterning of intermediate layer during the deposition of the intermediate layer by bringing a foil containing defined openings in direct contact with the surface of the base layer. In such a way, the material for the intermediate layer is only deposited on the base layer at the locations of the foil openings.

4. Screen printing

a. Screen print paste containing material for intermediate layer through a screen or stencil resembling desired pattern of intermediate layer on top of base layer.

### EXAMPLE 3

### Pafterning of PECVD coating

Various embodiments for patterning a PECVD coating are set forth below:

1. Lift-off

a. Deposit and pattern photoresist on top of surface of base layer

b. Vacuum deposit PECVD coating on top of base layer

c. Remove photoresist and thereby lifting off portions of PECVD coating, which were deposited on top of photoresist leaving patterned PECVD coating behind

2. Shadow masking:

patterning of PECVD coating during the deposition can be achieved by bringing a foil containing defined

openings in direct contact with the surface of the base layer or intermediate layer or both in such a way, that the PECVD coating is only deposited at the locations of the foil openings

3. Plasma Etching of PECVD Coating

Patterning of the PECVD coating can also be achieved by etching selected regions of the coating after deposition. In this dry etching mode, selected regions of the coating may be protected from the etching environment either by applying photoresist or covering these regions with a shadow mask. The etching environment may comprise reactive gas plasma using gases such as, without limitation, oxygen, a fluorine containing gas such as carbon tetrafluoride or sulphur hexafluoride, or mixtures of oxygen and the fluorine containing gas. The PECVD coating can also be selectively removed by ion milling or sputtering using an inert gas plasma or ion beam source.

The invention is illustrated by reference to the above embodiments. It should be appreciated however that the invention is not limited to these embodiments but is instead defined by the claims that follow.

That which is claimed:

1. A substrate having a chemically modified surface, said substrate comprising a base layer having a coating positioned thereon, the coating comprising a chemically crosslinked material comprising elements selected from the group consisting of (1) M, O, C, H, and N; wherein M is a metal selected from the group consisting of silicon, titanium, tantalum, germanium, boron, zirconium, aluminum, hafnium and yttrium; (2) M, O, H, and N wherein M is defined above, (3) C; (4) O, C, H, and N; and (5) M or C, and one of O, H, or N, wherein the chemically crosslinked material is terminated with at least one electrophilic or nucleophilic functional group.

2. The substrate according to claim 1, wherein M is silicon.

3. The substrate according to claim 2, wherein the chemically crosslinked composition comprises from about 30 to about 60 percent carbon and from about 10 to about 40 percent silicon.

4. The substrate according to claim 1, wherein the surface of the coating is terminated with at least one electrophilic functional group.

5. The substrate according to claim 4, wherein the at least one electrophilic functional group comprises one or more elements selected from the group consisting of carbon, hydrogen, nitrogen and silicon.

6. The substrate according to claim 5, wherein at least one biomolecule is adsorbed to the at least one electrophilic functional group.

7. The substrate according to claim 6, wherein the at least one biomolecule is selected from the group consisting of DNA, nucleic acids, proteins, enzymes, cells, viruses, and combinations thereof.

8. The substrate according to claim 1, wherein the surface of the coating is terminated with at least one nucleophilic functional group.

9. The substrate according to claim 8, wherein the at least one nucleophilic functional group comprises one or more elements selected from the group consisting of carbon, silicon, halogens, oxygen, hydrogen, nitrogen, sulfur, and phosphorus.

10. The substrate according to claim 8, wherein the surface of the coating is non-adsorbing with respect to biomolecules.

11. A substrate according to claim 1, wherein at least one portion of the surface of the coating is terminated with at least one electrophilic functional group and at least one other portion of the surface of the coating is terminated with at least one nucleophilic functional group.

12. The substrate according to claim 11, wherein the at least one portion of the surface of the coating terminated with at least one electrophilic functional group and at least one other portion of the surface of the coating terminated with at least one nucleophilic functional group are adjacent to one another.

13. The substrate according to claim 11, wherein the at least one nucleophilic functional group comprises one or more elements selected from the group consisting of carbon, silicon, halogens, oxygen, hydrogen, nitrogen, sulfur, and phosphorus.

14. The substrate according to claim 11, wherein the at least one electrophilic functional group comprises one or more elements selected from the group consisting of carbon, nitrogen, hydrogen, and silicon.

15. The substrate according to claim 11, wherein at least one biomolecule is adsorbed to the at least one electrophilic functional group.

16. The substrate according to claim 15, wherein the at least one biomolecule is selected from the group consisting of nucleic acids, proteins, enzymes, cells, viruses, and combinations thereof.

17. The substrate according to claim 1, wherein the coating is at least a fraction of a monolayer thick.

18. The substrate according to claim 1, wherein the base layer comprises a material selected from the group consisting of glass, plastics, elastomers, metals, and ceramics.

19. The substrate according to claim 1, further comprising an intermediate layer positioned between said base layer and said coating.

20. The substrate according to claim 19, wherein the intermediate layer comprises at least one material selected from the group consisting of indium tin oxide (ITO), tin oxide, titanium oxide, manganese oxide, lead oxide, gold, platinum, palladium, carbon, , silicon, germanium, zinc oxide, cadmium sulfide, titanium dioxide, gallium arsenide, combinations thereof, and alloys thereof.

21. A detection system comprising the substrate according to claim 1.

22. The detection system according to claim 21, wherein the detection system is selected from the group consisting of an electrochemical detection system, a chemical detection system, and an optical detection system.

23. The detection system according to claim 21, wherein the surface of the coating is terminated with at least one electrophilic group.

24. The detection system according to claim 23, wherein the at least one electrophilic group consists of one or more elements selected from the group consisting of carbon, nitrogen, hydrogen, and silicon.

25. The detection system according to claim 23, wherein at least one biomolecule is adsorbed to the at least one electrophilic group.

26. The detection system according to claim 25, wherein the at least one biomolecule is selected from the group consisting of nucleic acids, proteins, enzymes, cells, viruses, and combinations thereof.

27. The detection system according to claim 21, wherein the surface of the coating is terminated with at least one nucleophilic group.

28. The detection system according to claim 27, wherein the at least one nucleophilic group comprises one or more elements selected from the group consisting of carbon, silicon, halogens, oxygen, hydrogen, nitrogen, sulfur, and phosphorus.

29. The detection system according to claim 21, wherein the surface of the coating is non-adsorbing with respect to biomolecules.

30. The detection system according to claim 29, wherein the biomolecules are selected from the group consisting of nucleic acids, proteins, enzymes, cells, viruses, and combinations thereof.

31. A microfluidic system comprising the substrate according to claim 1.

32. The microfluidic system according to claim 32, wherein the system comprises at least one biofouling surface.

33. A method of chemically modifying a surface on a substrate, said method comprising:
exposing a composition comprising at least one precursor to an energy source to form a energized precursor;
promoting the energized precursor into an excited state to produce ionic materials;
depositing the ionic materials on a base layer such that the ionic materials form a coating thereon, the coating comprising a chemically crosslinked material comprising elements selected from the group consisting of (1) M, O, C, H, and N; wherein M is a metal selected from the group consisting of silicon, titanium, tantalum, germanium, boron, zirconium, aluminum, hafnium and yttrium; (2) M, O, H, and N wherein M is defined above, (3) C; (4) O, C, H, and N; and (5) M or C, and one of O, H, or N; and
treating the chemically crosslinked material such that the material is terminated with at least one electrophilic or nucleophilic functional group.

34. The method according to claim 33, wherein the precursor is selected from the group consisting of silanes, siloxanes, silazanes, hydrocarbons, metal organics, titanates, metal alkoxides, and combinations of the above.

35. The method according to claim 33, wherein the precursor is selected from the group consisting of tetrametylsilane, trimetylsilane, tetramethoxysilane, hexamethyl disilane, hexamethyl disiloxane, hexamethyl disilazane, methane, ethane, ethylene, tetraalkoxy titanates, and combinations thereof.

36. The method according to claim 33, wherein the energy source is selected from the group consisting of direct current, radio frequency, microwave, enhanced plasma, and a hollow cathode magnetron source.

37. The method according to claim 33, wherein said method is a Chemical Vapor Deposition (CVD).

38. The method according to claim 33, wherein said method is a Plasma Enhanced Chemical Vapor Deposition (PECVD).

39. The method according to claim 33, wherein the ionic materials comprise ionic fragments, free radicals, atoms, molecules, and mixtures thereof.

40. The method according to claim 33, wherein said treating step comprises treating the chemically crosslinked material such that the material is terminated with at least one electrophilic functional group

41. The method according to claim 40, wherein said treating step comprises a plasma treatment.

42. The method according to claim 33, wherein said treating step comprises treating the chemically crosslinked material such that the material is terminated with at least one nucleophilic functional group.

43. The method according to claim 42, wherein said treating step comprises a plasma treatment.
